(19) United States
(12) Patent Application Publication
(10) Pub. No.: US 2015/0335898 A1
(43) Pub. Date: Nov. 26, 2015

(54) SYSTEM AND METHOD FOR SIMULTANEOUS BURST AND TONIC STIMULATION

(71) Applicant: PACESETTER, INC., Sylmar, CA (US)

(72) Inventors: Mark Carlson, Calabasas, CA (US); Gene A. Bornzin, Simi Valley, CA (US); Melanie Goodman Keiser, McKinney, TX (US); John M. Murphy, Frisco, TX (US)

(73) Assignee: PACESETTER, INC., Sylmar, CA (US)

(21) Appl. No.: 14/284,299

(22) Filed: May 21, 2014

(51) Int. Cl. A61N 1/36

(52) U.S. Cl. CPC A61N 1/36171; A61N 1/36071; A61N 1/3615; A61N 1/36178

(57) ABSTRACT

A system and method for simultaneous burst and tonic stimulation of nerve tissue is provided. The system and method includes providing a lead with at least one stimulation electrode configured to be implanted at a target position proximate to nerve tissue of interest. The system and method further includes coupling the lead to an implantable pulse generator (IPG). The IPG generates current pulses that are delivered through blocking capacitors to the stimulation electrodes. The system and method further provides programming the IPG to deliver a first series of current pulses configured as a tonic stimulation waveform to the stimulation electrodes and to deliver a second series of current pulses configured as a burst stimulation waveform to the stimulation electrodes. The tonic and burst stimulation waveforms each include at least two current pulses with different amplitude polarities.

### FIELD OF THE INVENTION

Embodiments of the present disclosure generally relate to neurostimulation (NS) systems, and more particularly to generating simultaneous burst and tonic stimulation signals.

### BACKGROUND OF THE INVENTION

NS systems are devices that generate electrical pulses and deliver the pulses to nervous tissue to treat a variety of disorders. For example, spinal cord stimulation has been used to treat chronic and intractable pain. Another example is deep brain stimulation, which has been used to treat movement disorders such as Parkinson's disease and affective disorders such as depression. While a precise understanding of the interaction between the applied electrical energy and the nervous tissue is not fully appreciated, it is known that application of electrical pulses to certain regions or areas of nerve tissue can effectively mask certain types of pain transmitted from regions, increase the production of neurotransmitters, or the like. For example, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

The effectiveness of the NS of nervous tissue may be dependent on the amplitude or frequency of the electrical pulses. For example, a tonic stimulation waveform may be more effective to relieve foot pain of a patient than a burst stimulation waveform. In another example, the patient also suffers from back pain, which the burst stimulation waveform may be more effective to relieve than the tonic stimulation waveform. Previous NS systems were only able to generate a certain type of stimulation waveform (e.g., either tonic stimulation or burst stimulation waveform). Thus, the patient described in the above example would require two NS systems to relieve both the foot and back pain. In addition, it is possible that burst and tonic stimulation have different effectiveness for specific aspects of pain. For instance, burst may more effectively treat perception or reaction to pain (i.e. catastrophization) and that tonic stimulation may more effectively relieve the pain itself.

Accordingly, NS systems have been proposed to produce a burst stimulation and a tonic stimulation waveform from electrodes on a lead. For example, the proposed NS system that produces the pulses described in FIG. 7 of U.S. Pat. No. 8,364,273, entitled, “COMBINATION OF TONIC AND BURST STIMULATION TO TREAT NEUROLOGICAL DISORDERS,” which is expressly incorporated herein by reference. The proposed NS system may be beneficial to the patient in the above examples. However, the proposed NS system does not account for charge balancing the electrodes, for example, after the tonic stimulation. Maintaining charge balance on NS electrodes is important because over the life of the electrodes tens or hundreds of amp-hours may be passed, which can damage the electrodes. Moreover, the proposed NS system requires a temporal limitation on the tonic stimulation to occur only after the burst stimulation, thus, restricting the frequency of the tonic stimulation.

### SUMMARY

In accordance with one embodiment, a method for simultaneous burst and tonic stimulation of nerve tissue is provided. The method includes providing a lead having at least one stimulation electrode on the lead to be implanted at a target position proximate to nerve tissue of interest, and coupling the lead to an implantable pulse generator (IPG). The IPG generates current pulses that are delivered through blocking capacitors to the stimulation electrodes. The method further provides programming the IPG to deliver a first series of current pulses configured as a tonic stimulation waveform to the stimulation electrodes and to deliver a second series of current pulses configured as a burst stimulation waveform to the stimulation electrodes. The tonic stimulation waveform and the burst stimulation waveform each include at least two current pulses with different amplitude polarities.

In an embodiment, a system for simultaneous burst and tonic stimulation is provided. The system includes a lead having at least one stimulation electrode. The lead is configured to be implanted to a target position proximate to or within nerve tissue of interest. The system also includes an implantable pulse generator (IPG) that is coupled to the lead. The IPG is configured to deliver a first and second series of current pulses through blocking capacitors to the stimulation electrodes. The first series of current pulses are configured as a tonic stimulation waveform and delivered to the stimulation electrodes. The second series of current pulses are configured as a burst stimulation waveform and delivered to the stimulation electrodes. The tonic stimulation waveform and the burst stimulation waveform each include at least two current pulses having different amplitude polarities.

### BRIEF DESCRIPTION OF THE DRAWINGS

FIG. 1 illustrates a neurostimulation system, according to an embodiment of the present disclosure.

FIG. 2 illustrates a schematic diagram of the neurostimulation system, according to an embodiment of the present disclosure.

FIG. 3a illustrates a graphical representation of a current amplitude across blocking capacitors during two pulses, according to an embodiment of the present disclosure.

FIG. 3b illustrates a graphical representation of a voltage potential across blocking capacitors during two pulses, according to an embodiment of the present disclosure.

FIG. 4 illustrates a lead proximate to two stimulation targets, according to an embodiment of the present disclosure.

FIG. 5a illustrates a lead proximate to two stimulation targets, according to an embodiment of the present disclosure.

FIG. 5b illustrates a lead proximate to two stimulation targets, according to an embodiment of the present disclosure.

FIG. 6a illustrates a graphical representation of a burst stimulation waveform and a tonic stimulation waveform, according to an embodiment of the present disclosure

FIG. 6b illustrates a graphical representation of a burst stimulation waveform and a tonic stimulation waveform, according to an embodiment of the present disclosure

FIG. 7a illustrates a graphical representation of a pulse, according to an embodiment of the present disclosure

FIG. 7b illustrates a graphical representation of a subdivided pulse from the pulse in FIG. 7a, according to an embodiment of the present disclosure.

FIG. 8 illustrates a graphical representation of a chopped burst and tonic stimulation waveform, according to an embodiment of the present disclosure

FIG. 9 is a flowchart of a method for stimulating a burst and tonic stimulation of nerve tissue of a patient.

FIG. 10 illustrates a schematic diagram of the neurostimulation system, according to an embodiment of the present disclosure.

FIG. 11 illustrates a graphical representation of two chopped burst stimulation waveforms and a tonic stimulation waveform, according to an embodiment of the present disclosure.

### DETAILED DESCRIPTION

Embodiments described herein include neurostimulation (NS) systems and methods for generating simultaneous tonic and burst stimulation waveforms using the same. The NS lead may be configured to be inserted into a space or cavity of a patient and positioned adjacent to nervous tissue of interest. In certain embodiments, the NS lead includes wireless leads that are positioned entirely within an epidural space of a spinal column.

While multiple embodiments are described, still other embodiments of the described subject matter will become apparent to those skilled in the art from the following detailed description and drawings, which show and describe illustrative embodiments of disclosed inventive subject matter. As will be realized, the inventive subject matter is capable of modifications in various aspects, all without departing from the spirit and scope of the described subject matter. Accordingly, the drawings and detailed description are to be regarded as illustrative in nature and not restrictive.

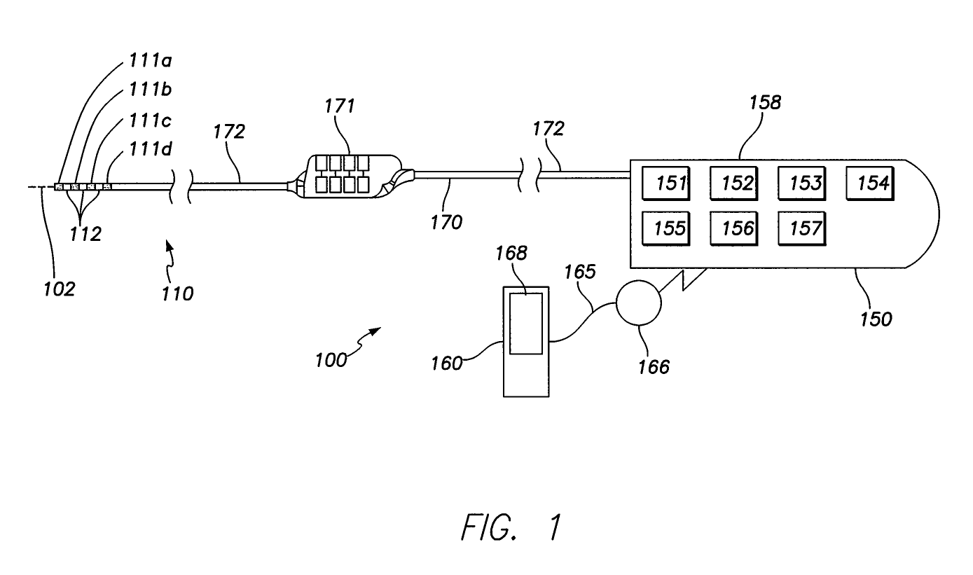

FIG. 1 depicts an NS system 100 that generates electrical pulses for application to tissue of a patient according to one embodiment. For example, the NS system 100 may be adapted to stimulate spinal cord tissue, peripheral nerve tissue, deep brain tissue, cortical tissue, cardiac tissue, digestive tissue, pelvic floor tissue, or any other suitable nerve tissue of interest within a patient's body.

The NS system 100 includes an implantable pulse generator (IPG) 150 that is adapted to generate electrical pulses for application to tissue of a patient. The IPG 150 typically comprises a metallic housing or can 158 that encloses a controller 151, pulse generating circuitry 152, a charging coil 153, a battery 154, a far-field and/or near field communication circuitry 155, battery charging circuitry 156, switching circuitry 157, and the like. The controller 151 typically includes a microcontroller or other suitable processor for controlling the various other components of the device. Software code is typically stored in memory of the IPG 150 for execution by the microcontroller or processor to control the various components of the device.

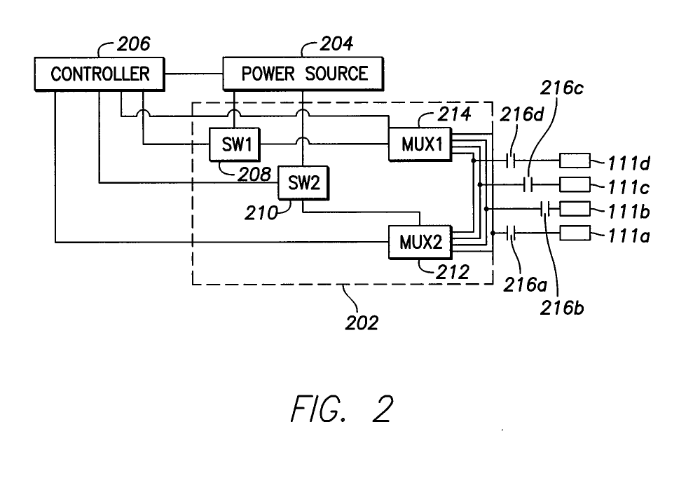

The IPG 150 may comprise a separate or an attached extension component 170. If the extension component 170 is a separate component, the extension component 170 may connect with the “header” portion of the IPG 150 as is known in the art. If the extension component 170 is integrated with the IPG 150, internal electrical connections may be made through respective conductive components. Within the IPG 150, electrical pulses are generated by the pulse generating circuitry 152 and are provided to the switching circuitry 157. The switching circuitry 157 connects to outputs of the IPG 150. Electrical connectors (e.g., “Bal-Seal” connectors) within the connector portion 171 of the extension component 170 or within the IPG header may be employed to conduct various stimulation pulses. The terminals of one or more leads 110 are inserted within connector portion 171 or within the IPG header for electrical connection with respective connectors. Thereby, the pulses originating from the IPG 150 are provided to the leads 110. The pulses are then conducted through the conductors of the lead 110 and applied to tissue of a patient via stimulation electrodes 111a-d that are coupled to blocking capacitors (e.g., blocking capacitors 216a-d in FIG. 2). Any suitable known or later developed design may be employed for connector portion 171.

The stimulation electrodes 111a-d may be positioned along a horizontal axis 102 of the lead 110, and are angularly positioned about the horizontal axis 102 so the stimulation electrodes 111a-d do not overlap. The stimulation electrodes 111a-d may be in the shape of a ring such that each stimulation electrode 111a-d continuously covers the circumference of the exterior surface of the lead 110. Each of the stimulation electrodes 111a-d are separated by non-conducting rings 112, which electrically isolate each stimulation electrode 111a-d from an adjacent stimulation electrode 111a-d. The non-conducting rings 112 may include one or more insulative materials and/or biocompatible materials to allow the lead 110 to be implantable within the patient. Non-limiting examples of such materials include polyimide, polyetheretherketone (PEEK), polyethylene terephthalate (PET) film (also known as polyester or Mylar), polytetrafluoroethylene (PTFE) (e.g., Teflon), or parylene coating, polyether bloc amides, polyurethane. The stimulation electrodes 111a-d may be configured to emit the pulses in an outward radial direction proximate to or within a stimulation target. Additionally or alternatively, the stimulation electrodes 111a-d may be in the shape of a split or non-continuous ring such that the pulse may be directed in an outward radial direction adjacent to the stimulation electrodes 111a-d. Examples of a fabrication process of the stimulation electrodes 111a-d is disclosed in U.S. patent application Ser. No. 12/895,096, entitled, “METHOD OF FABRICATING STIMULATION LEAD FOR APPLYING ELECTRICAL STIMULATION TO TISSUE OF A PATIENT,” which is expressly incorporated herein by reference.

It should be noted the stimulation electrodes 111a-d may be in various other formations, for example, in a planar formation on a paddle structure as disclosed in U.S. Provisional Application No. 61/791,288, entitled, “PADDLE LEADS FOR NEUROSTIMULATION AND METHOD OF DELIVERYING THE SAME,” which is expressly incorporated herein by reference.

The lead 110 may comprise a lead body 172 of insulative material about a plurality of conductors within the material that extend from a proximal end of lead 110, proximate to the IPG 150, to its distal end. The conductors electrically couple a plurality of the stimulation electrodes 111a-d to a plurality of terminals (not shown) of the lead 110. The terminals are adapted to receive electrical pulses and the stimulation electrodes 111a-d are adapted to apply the pulses to the stimulation target of the patient. Also, sensing of physiological signals may occur through the stimulation electrodes 111, the conductors, and the terminals. It should be noted that although the lead 110 is depicted with four stimulation electrodes 111a-d, the lead 110 may include any suitable number of stimulation electrodes 111a-d (e.g., less than four, more than four) as well as terminals, and internal conductors. Additionally or alternatively, various sensors (e.g., a position detector, a radiopaque fiducial) may be located near the distal end of the lead 110 and electrically coupled to terminals through conductors within the lead body 172.

Although not required for all embodiments, the lead body 172 of the lead 110 may be fabricated to flex and elongate upon implantation or advancing within the tissue (e.g., nervous tissue) of the patient towards the stimulation target and movements of the patient during or after implantation. By fabricating the lead body 172, according to some embodiments, the lead body 172 or a portion thereof is capable of elastic elongation under relatively low stretching forces. Also, after removal of the stretching force, the lead body 172 may be capable of resuming its original length and profile. For example, the lead body may stretch 10%, 20%, 25%, 35%, or even up or above to 50% at forces of about 0.5, 1.0, and/or 2.0 pounds of stretching force. Fabrication techniques and material characteristics for “body compliant” leads are disclosed in greater detail in U.S. Provisional Patent Application No. 60/788,518, entitled “Lead Body Manufacturing,” which is expressly incorporated herein by reference.

For implementation of the components within the IPG 150, a processor and associated charge control circuitry for an IPG is described in U.S. Pat. No. 7,571,007, entitled “SYSTEMS AND METHODS FOR USE IN PULSE GENERATION,” which is expressly incorporated herein by reference. Circuitry for recharging a rechargeable battery (e.g., battery charging circuitry 156) of an IPG using inductive coupling and external charging circuits are described in U.S. Pat. No. 7,212,110, entitled “IMPLANTABLE DEVICE AND SYSTEM FOR WIRELESS COMMUNICATION,” which is expressly incorporated herein by reference.

An example and discussion of “constant current” pulse generating circuitry (e.g., pulse generating circuitry 152) is provided in U.S. Patent Publication No. 2006/0170486 entitled “PULSE GENERATOR HAVING AN EFFICIENT FRACTIONAL VOLTAGE CONVERTER AND METHOD OF USE,” which is expressly incorporated herein by reference. One or multiple sets of such circuitry may be provided within the IPG 150. Different pulses on different stimulation electrodes 111a-d may be generated using a single set of the pulse generating circuitry 152 using consecutively generated pulses according to a “multi-stimset program” as is known in the art. Complex pulse parameters may be employed such as those described in U.S. Pat. No. 7,228,179, entitled “Method and apparatus for providing complex tissue stimulation patterns,” and International Patent Publication Number WO 2001/093953 A1, entitled “NEUROMODULATION THERAPY SYSTEM,” which are expressly incorporated herein by reference. Alternatively, multiple sets of such circuitry may be employed to provide pulse patterns (e.g., tonic stimulation waveform, burst stimulation waveform) that include generated and delivered stimulation pulses through various stimulation electrodes of one or more leads 111a-d as is also known in the art. Various sets of parameters may define the pulse characteristics and pulse timing for the pulses applied to the various stimulation electrodes 111a-d as is known in the art. Although constant current pulse generating circuitry is contemplated for some embodiments, any other suitable type of pulse generating circuitry may be employed such as constant voltage pulse generating circuitry.

A controller device 160 may be implemented to charge/recharge the battery 154 of the IPG 150 (although a separate recharging device could alternatively be employed) and to program the IPG 150 on the pulse specifications while implanted within the patient. Although, in alternative embodiments separate programmer devices may be employed for charging and/or programming the NS system 100. The controller device 160 may be a processor-based system that possesses wireless communication capabilities. Software may be stored within a non-transitory memory of the controller device 160, which may be executed by the processor to control the various operations of the controller device 160. A “wand” 165 may be electrically connected to the controller device 160 through suitable electrical connectors (not shown). The electrical connectors may be electrically connected to a telemetry component 166 (e.g., inductor coil, RF transceiver) at the distal end of wand 165 through respective wires (not shown) allowing bi-directional communication with the IPG 150. Optionally, in some embodiments, the wand 165 may comprise one or more temperature sensors for use during charging operations.

The user may initiate communication with the IPG 150 by placing the wand 165 proximate to the NS system 100. Preferably, the placement of the wand 165 allows the telemetry system of the wand 165 to be aligned with the far-field and/or near field communication circuitry 155 of the IPG 150. The controller device 160 preferably provides one or more user interfaces 168 (e.g., touchscreen, keyboard, mouse, buttons, or the like) allowing the user to operate the IPG 150. The controller device 160 may be controlled by the user (e.g., doctor, clinician) through the user interface 168 allowing the user to interact with the IPG 150. The user interface 168 may permit the user to move electrical stimulation along and/or across one or more of the lead(s) 110 using different stimulation electrode 111a-d combinations, for example, as described in U.S. Patent Application Publication No. 2009/0326608, entitled “METHOD OF ELECTRICALLY STIMULATING TISSUE OF A PATIENT BY SHIFTING A LOCUS OF STIMULATION AND SYSTEM EMPLOYING THE SAME,” which is expressly incorporated herein by reference.

Also, the controller device 160 may permit operation of the IPG 150 according to one or more stimulation programs to treat the patient. Each stimulation program may include one or more sets of stimulation parameters of the pulse including pulse amplitude, pulse width, pulse frequency or inter-pulse period, pulse repetition parameter (e.g., number of times for a given pulse to be repeated for respective stimset during execution of program), biphasic pulses, monophasic pulses, etc. The IPG 150 modifies its internal parameters in response to the control signals from the controller device 160 to vary the stimulation characteristics of the stimulation pulses transmitted through the lead 110 to the tissue of the patient. NS systems, stimsets, and multi-stimset programs are discussed in PCT Publication No. WO 01/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are expressly incorporated herein by reference.

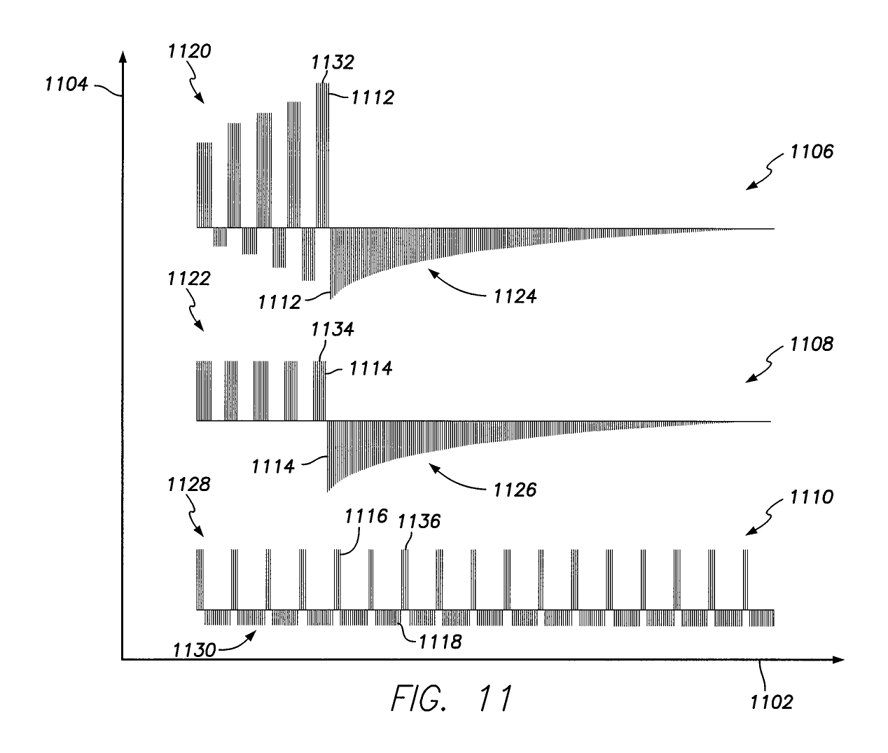

FIG. 2 is a basic schematic diagram of switching circuitry 202 for an embodiment of an NS system. The switching circuitry 202 (e.g., the switching circuitry 157) may be electrically coupled to a controller 206 (e.g., the controller 151), a power source 204 (e.g., battery 154), and a plurality of blocking capacitors 216a-d. The switching circuitry 202 is shown with two electrical switches, a switch1 208 and a switch2 210. The switches 208 and 210 are electrically coupled to two multiplexers, a MUX1 214 and a MUX2 212. It should be noted that the switching circuitry 202 may be generally characterized as switch arrays (e.g., plurality FETS, relays), switch matrixes, or the like. Thereby, in alternative embodiments the switching circuitry 202 may include more or less switches (e.g., more than two, less than two) than illustrated in FIG. 2. Additionally, the switching circuitry may include more or less multiplexers (e.g., more than two, less than two) than illustrated in FIG. 2. Optionally the switching circuitry 202 may be integrated within the controller 206. Optionally, the switching circuitry 202 (FIG. 2) may be electrically coupled to a can (e.g., the can 158, 1014) as described regarding to FIG. 10.

The switch1 208 and switch2 210 are electrically coupled to a power source 204 (e.g., the battery 154, boost converter). The power source 204 provides a direct current or voltage contact for the switch1 208 and switch2 210. The switch1 208 and switch2 210 are also electrically coupled to a common ground (not shown) for the NS system. The common ground provides a return path for electric current for the NS system. The switch1 208 and switch2 210 may direct current or form electrical current paths from the power source 204 and/or the common ground to the multiplexers 212 and 214 by electrically coupling to one of the contacts (e.g., the power source 204, the common ground). For example, the switch1 208 may electrically couple the power source 204 to the MUX1 214 and the switch2 210 may electrically couple the MUX2 212 to the common ground. Thereby, the MUX 214 may receive current or voltage from the power source 204.

The multiplexers 212 and 214 are each electrically coupled to a plurality of blocking capacitors 216a-d through conducting paths or wires 218. Each blocking capacitor 215a-d is coupled to a corresponding stimulation electrode 111a-d. The multiplexers 212 and 214 each may select or electrically couple one or more of the blocking capacitors 216a-d to the switches 208 and 210. Continuing from the above example configuration of switch1 208 and switch2 210, MUX1 214 selects the blocking capacitor 216d and MUX2 212 selects the blocking capacitor 216a. Thereby, the blocking capacitor 216d is electrically coupled to the power source 204, and the blocking capacitor 216a is electrically coupled to the common ground. It should be understood that the multiplexers 212 and 214 may select multiple (e.g., more than one) blocking capacitors 216a-d.

The switching circuitry 202 and the power source 204 are controlled by the controller 206 to configure pulses that are emitted from the NS system through the stimulation electrodes 111a-d. The controller 206 controls or adjust the amount of current or voltage supplied to the switches 208 and 210 by instructing the amount of current or voltage supplied by the power source 204 to the switches 208 and 210. Additionally, the controller 206 may instruct at least one of the switches 208 and 210 to electrically couple to one of the multiplexers 212 and 214. Likewise, the controller 206 may instruct the multiplexers 212 and 214 to select at least one of the blocking capacitors 216a-d.

For example, the NS system 100 is programmed to emit a 2 milliampere (mA) pulse. The pulse is programmed to be discharged from the stimulation electrode 111a in an anode state or when the stimulation electrode 111a is electrically coupled to the power source 204 via the MUX11 214, and grounded by the stimulation electrode 111d in a cathode state or when the stimulation electrode 111d is electrically coupled to the common ground via the MUX2 212. The controller 206 may instruct the power source 204 to supply a 2 mA supply current to the switches 208 and 210. The controller 206 may instruct the switch1 208 to direct current or electrically couple the MUX1 214 to the power source 204, and have the MUX1 214 select the blocking capacitor 216a that is coupled to the stimulation electrode 111a. The controller 206 may further instruct the switch2 210 to electrically couple the MUX2 212 to the common ground, and have the MUX2 212 select the blocking capacitor 216d that is coupled to the stimulation electrode 111d.

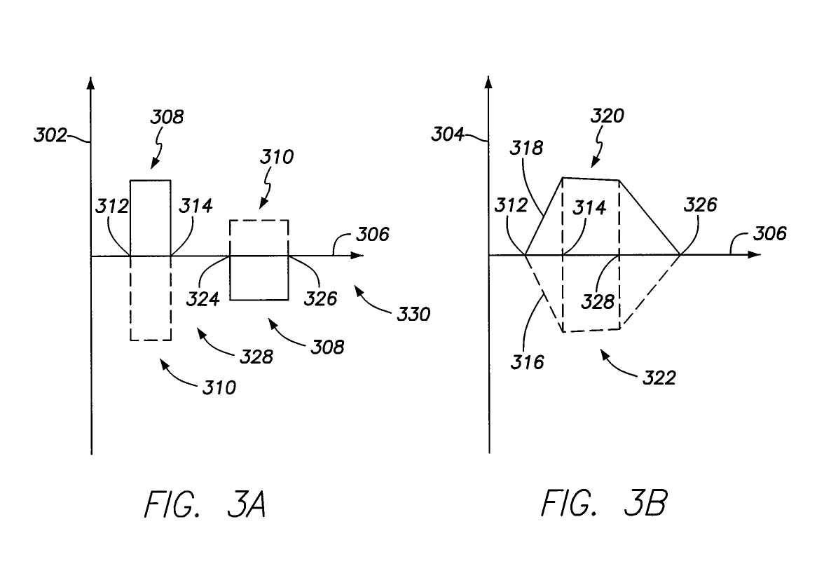

FIGS. 3a-b illustrate a graphical representation of the electrical characteristic of the blocking capacitors 216a and 216d during two pulses 328 and 330 when a pulse is emitted from the NS system 100. The horizontal axes 306 represent time. The vertical axes 302 and 304 represent current and the voltage potential, respectively, across the blocking capacitor 216a and 216d. At time 312, the stimulation electrode 111a and the stimulation electrode 111d are set by the controller 206 to the anode and cathode state, respectively. In FIG. 3a, a current 308 represents the electrical current flow across the blocking capacitor 216a supplied by the power source 204 through the MUX1 214 and the switch1 208. A current 310 represents the electrical current across the blocking capacitor 216d, which is electrically coupled to the common ground through the MUX2 212 and the switch2 210. The amplitude of the currents 308 and 310 are approximately the supply current (e.g., 2 mA) from the power source 204 configured by the controller 206. It should be noted that the difference in amplitude polarities of the currents 308 and 310 represent the opposing direction of electric charge or current flow in relation to the NS system 100 from both electrodes 111a and 111d as the pulse is emitted from the stimulation electrode 111a. At time 314, the stimulation electrodes 111a and 111d may be configured in an inoperative state (in which case the stimulation electrode is not used for transmitting energy, i.e., is inactive or open) reducing the currents 308 and 310 to near zero.

FIG. 3b shows that during the pulse 328, from time 312 to 314 (e.g., 1 millisecond (ms)), a voltage potential builds on each blocking capacitor 216a and 216d. The voltage potentials build at a linear rate having slopes 316 and 318 (_{dt}^{dV(t)}) and are related to the corresponding currents 308 and 310 (I(t)), respectively, and the capacitance (C) of each blocking capacitor 216a and 216d, as shown in Equation 1.

$$I ( t ) = C \frac{d V ( t )}{d t} \qquad ( Equation 1 )$$

After the pulse 328 (at time 314), the voltage potentials 320 and 322 is stored on the blocking capacitors 216a and 216d. It should be noted that the voltage potentials 320 and 322 may slowly dissipate due to leakage or imperfection of the blocking capacitors 216 and 216d as shown in FIG. 3b during the time between time 324 and 314. It should be noted that continual pulses (e.g., stimulation waveforms) emitted with the same amplitude polarity and stimulation electrodes 111a and 111d without dissipating the voltage potentials 320 and 322, may continually increase the voltage potential 320 and 322 across the blocking capacitors 216a and 216d. The remaining voltage potential 320 and 322, after each stimulation waveform, may create a charge imbalance between the electrodes 111a and 111d. To dissipate the voltage potential across the blocking capacitors 216a and 216d, the polarity of the stimulation electrodes 111a and 111d may be reversed. The discharge of the blocking capacitors 216a and 216d may return the voltage potentials 320 and 322 to approximately the same level before the pulse 328. Maintaining a charge balance on the stimulation electrodes 111a and 111d for each stimulation waveform.

For example, at time 324 the controller 206 may cause the stimulation electrode 111a to enter the cathode state by instructing the switch1 208 to electrically couple the MUX1 214 to the common ground. The controller 206 may cause the stimulation electrode 111d to enter the anode state by instructing the switch2 210 to electrically couple the MUX2 212 to the power source 204. The reversal of the states of the stimulation electrodes 111a and 111d switches the polarities of the currents 308 and 310 during the second pulse (e.g., discharge pulse) 330. During the pulse 330, the voltage potentials 320 and 322 across the blocking capacitors 216a and 216d decrease. At time 314, the stimulation electrodes 111a and 111b may be configured in an inoperative state (in which case the stimulation electrode is not used for transmitting energy, i.e., is inactive or open) reducing the currents 308 and 310 to near zero.

Optionally, the controller 206 may instruct the power source 204 to adjust the amplitude of the pulse 330 relative to the previous pulse 328. Further, the controller 206 may adjust the duration of the pulse 330. It should be noted that the adjustments to the amplitude and duration are inversely related such that the integral of the amplitudes of the pulse 328 over the time period between time 324 and 326 is approximate to the voltage potentials 320 and 322 at time 324.

For example, FIG. 3a illustrates the change in polarities of the currents 308 and 310 during the pulse 330 from time 324 to time 326. It should be noted that the amplitude of the currents 308 and 310 during the pulse 330 is reduced relative to the amplitude of the pulse 328 (e.g., from 2 mA to 1 mA), and the duration of the pulse 330 is also increased relative to the pulse 328 (e.g., from 1 ms to 2 ms). The adjusted amplitude of the pulse 330 reduces the rate that the voltage potentials 320 and 322 are dissipated across the blocking capacitors 216a and 216d. However, the increased duration of the pulse 330 allows for the voltage potentials 320 and 322 to dissipate to near zero at time 326 resulting in a charge balance between times 312 and 326.

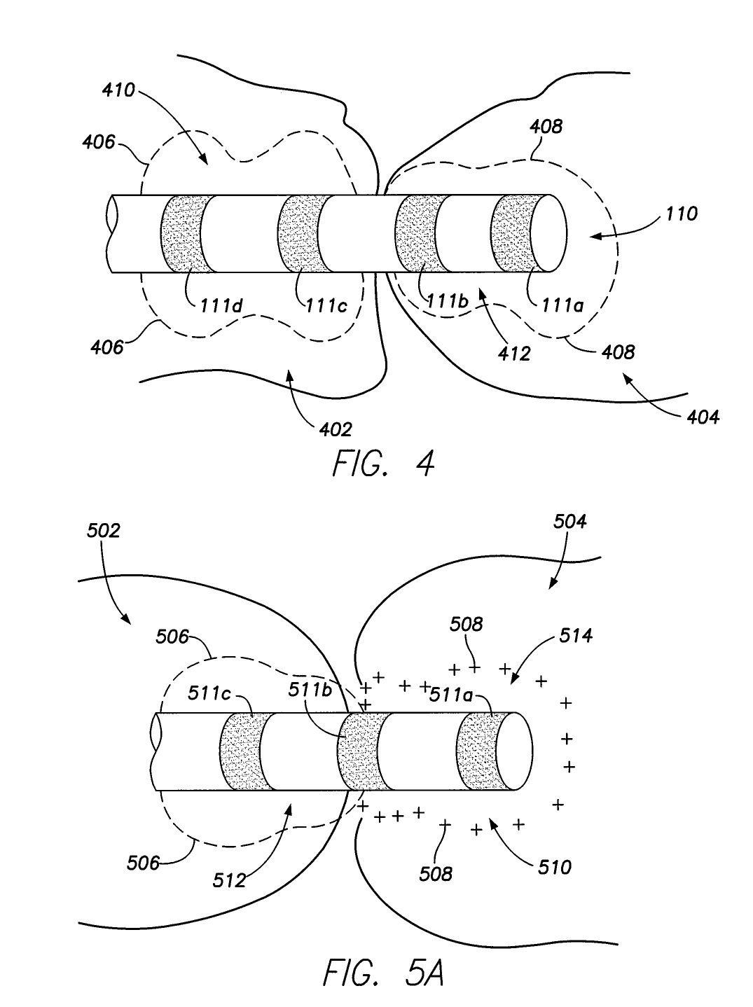

FIG. 4 illustrates the lead 110 positioned proximate or within the stimulation targets 402 and 404, for example, nerve tissue for spinal cord stimulation (e.g., 402) and peripheral nerve tissue (e.g., 404), such that the surface area of the lead 110 is proximate to both of the stimulation targets 402 and 404. The position of the lead 110 allows a first sub-set of stimulation electrodes 410 (e.g., the stimulation electrodes 111c-d) and a second sub-set of stimulation electrodes 412 of stimulation electrodes (e.g., the stimulation electrodes 111a-b), that have energy trajectories 406 and 408 overlap separate stimulation targets 402 and 404, respectively. The energy trajectories 406 and 408 may represent an area or distance from the first and second sub-sets of stimulation electrodes 410 and 412, respectively, to the stimulation targets 402 and 404 that the electrical pulse emitted by the stimulation electrodes 410 and 412 may be propagated through the surrounding tissue and stimulate the stimulation targets 402 and 404. The separation of the energy trajectories 406 and 408 allows one of the sub-sets of stimulation electrodes 410 and 412 to stimulate a corresponding simulation target 402 and 404 without affecting the adjacent stimulation target 404 and 402, respectively.

The area or distance from the energy trajectories 406 and 408 may be increased or decreased by adjusting the amplitude of the pulse. It should be noted that as the pulses traverse through the tissue surrounding the lead 110 away from the stimulation electrodes 111a-d, the amplitude of the pulse decreases due to the resistance of the surrounding tissue. The change in the pulse amplitude may reduce the effectiveness of the pulse in stimulating the stimulation targets 402 and 404. For example, the pulses emitted from the second sub-set of stimulation electrodes 412 may be configured to have a pulse amplitude of 10 mA. Preferably, the stimulation targets may be within 5.0 mm of the second sub-set of stimulation electrodes 412 to effectively stimulate the stimulation target 404 by the pulse. It should be noted, that increasing the pulse amplitude may increase the effective distance available as an option between the second sub-set of stimulation electrodes 412. Conversely, when the pulse amplitude is decreased the effective distance may also decrease. For example, an electrode that delivers a pulse having a pulse amplitude of 1 mA would preferably be closer to the stimulation target 404 relative to an electrode that delivers a pulse having a pulse amplitude of 10 mA.

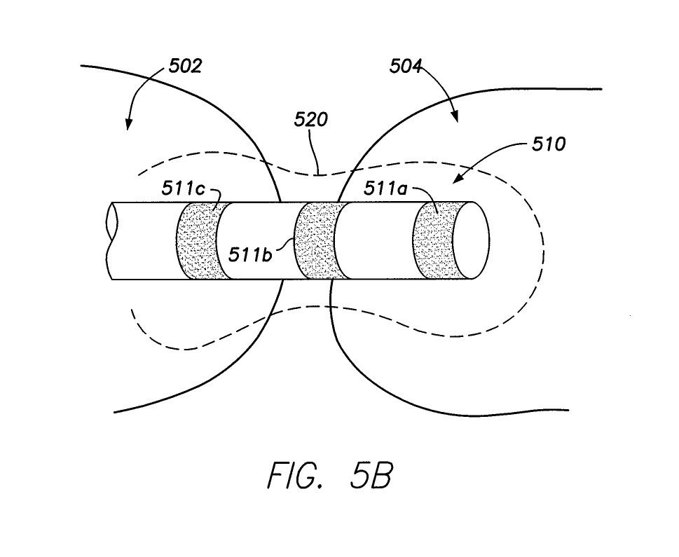

Optionally, as shown in FIG. 5a, a lead 510 may be positioned such that a first sub-set of stimulation electrodes 512 (e.g., the stimulation electrodes 511b-c) and a second sub-set of stimulation electrodes 514 of stimulation electrodes (e.g., the stimulation electrodes 511a-b) have a single unique stimulation electrode (e.g., 511a and 511c). Each sub-set of stimulation electrodes 512 and 514, similar to FIG. 4, have energy trajectories 506 and 508 that overlap separate stimulation targets 502 and 504, respectively. Each sub-set of stimulation electrodes (e.g., 410, 412, 512, 514) may emit a burst and/or tonic stimulation waveform.

Additionally or alternatively, the stimulation electrodes are not divided into subset. FIG. 5b illustrates the stimulation electrodes 511a-c of the lead 510 with a common energy trajectory 520. The stimulation electrodes 511a-c each may emit a burst and/or tonic stimulation waveforms, using the methods as discussed further below.

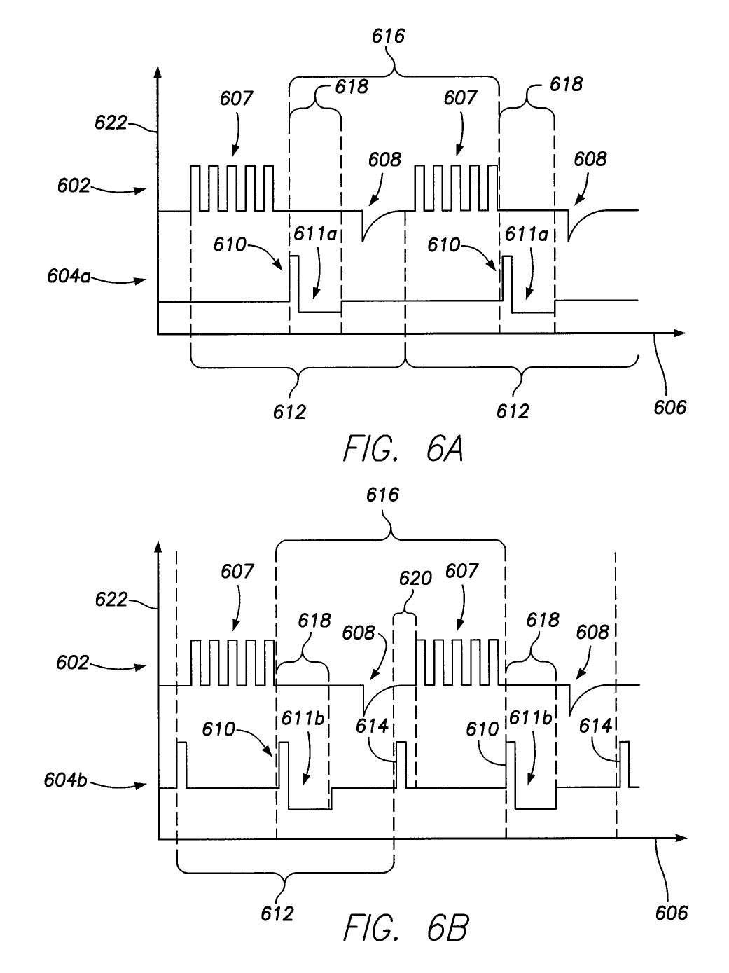

FIGS. 6a-b illustrates a graphical representation of a burst stimulation waveform 602 and first and second tonic stimulation waveforms 604a-b simultaneously emitted from the first-subset of stimulation electrodes 410 and the second sub-set of stimulation electrodes 412, respectively. The horizontal axis 606 represents time, and the vertical axis 622 may represent current amplitude. The burst stimulation waveform 602 may be repeated over a set period 612 of, for example, 25 ms or a frequency of 40 Hz. The burst stimulation waveform 602 includes a series of burst pulses 607. For example, one burst may have five pulses with approximately the same amplitude. Each of the pulses may have a pulse width of, for example, 2 ms such that the burst pulses 607 have a frequency of 500 hertz (Hz). It should be noted that although the burst pulses 607 are shown in FIGS. 6a-b with five pulses, in alternative embodiments the burst pulses 607 may include more or fewer pulses (e.g., less than five pulses, more than five pulses). Additionally or alternatively, the frequency of the burst pulses 607 may be greater than or less than 500 Hz. Optionally, the amplitude of the burst pulses 607 may vary such that at least one of the pulses within the series of burst pulses 607 has a different amplitude (e.g., burst pulses 810 in FIG. 8).

The burst stimulation waveform 602 also includes a recharge pulse 608. The recharge pulse 608, similar to the pulse 330, has a different polarity than the burst pulses 607 to maintain charge balance for the first sub-set of stimulation electrodes 410 (e.g., stimulation electrodes 111c-d). The recharge pulse 608 is illustrated after the burst pulses 607. However, in alternative embodiments the recharge pulse 608 may occur before the burst pulses 607. Optionally, the recharge pulse 608 may be before and/or after a plurality of burst pulses 607.

The tonic stimulation waveform 604a is shown with a set period 616, for example, of 25 ms or a frequency of 40 Hz. The tonic stimulation waveform 604a includes a tonic pulse 610 and a recharge pulse 611a. The recharge pulse 611a, similar to the pulse 330, has a different polarity than the tonic pulse 607 to maintain charge balance for the second sub-set of stimulation electrodes 412 (e.g., stimulation electrodes 111a-b). The tonic stimulation waveform 604a times pulses 610, 611a to be temporally offset with respect to pulses 607, 608 of the burst stimulation waveform 602. Thereby, the tonic pulse 610 and the recharge pulse 611a do not occur during one of the burst pulses 607 or the recharge pulse 608. For example, during a period 618 the tonic pulse 610 and the recharge pulse 611a are emitted by the second sub-set of stimulation electrodes 412. However, during the period 618 there are no pulses (e.g., burst pulses 607, recharge pulse 608) emitted by the first sub-set of stimulation electrodes 410. During the period 618, the waveform 602 maintains a non-burst or neutral state. The pulses 610, 611a occur during an inter-pulse-burst gap between bursts of pulses 607.

Optionally, the tonic stimulation waveform 604a occurs within the burst stimulation waveform 602 such that burst pulses 607 of the burst stimulation waveform 602 occurs before and after the tonic stimulation waveform 604a.

The temporal offset between the pulses of the two waveforms 604a and 602 allow each waveform 604a and 602 to be adjusted independently (e.g., amplitude, duration) without compromising the alternative waveform. For example, FIG. 6b illustrates an adjusted tonic stimulation waveform 604b with an additional tonic pulse 614 and a recharge pulse 611b with an increased amplitude relative to the tonic stimulation waveform 604a. The additional tonic pulse 614 increases the frequency of stimulation (e.g., the number of tonic pulses 610 and 614 within the set period 616) of the adjusted tonic stimulation waveform 604b compared to the tonic stimulation waveform 604a, for example, from a frequency of 40 Hz to approximately 80 Hz. The additional tonic pulse 614 occurs during a period 620 in which no pulses (e.g., burst pulses 607, recharge pulse 608) are emitted by the first sub-set of stimulation electrodes 410. The amplitude of the recharge pulse 611b is increased to account for the additional tonic pulse 614 to maintain charge balance of the second sub-set of stimulation electrodes 412.

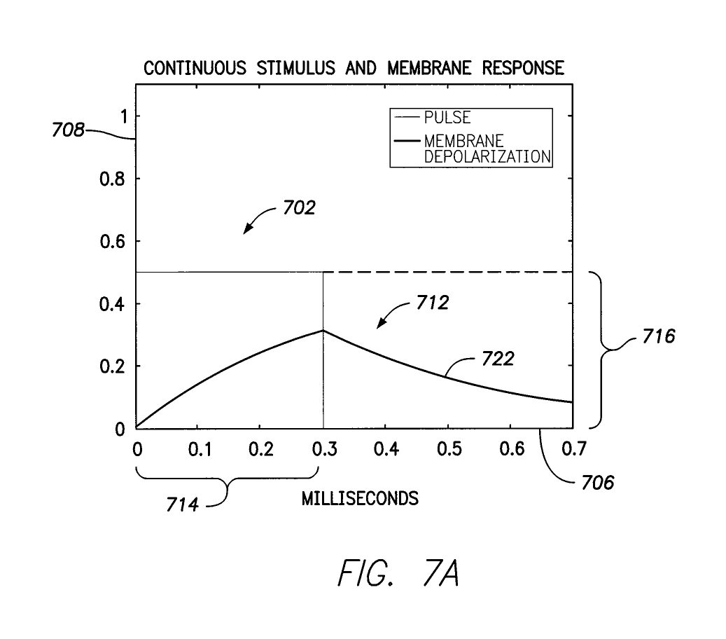

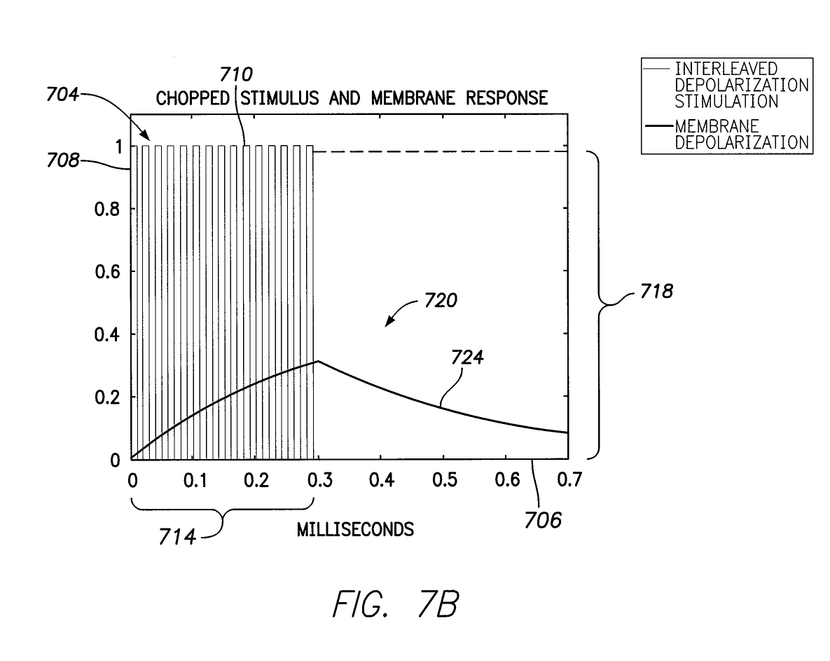

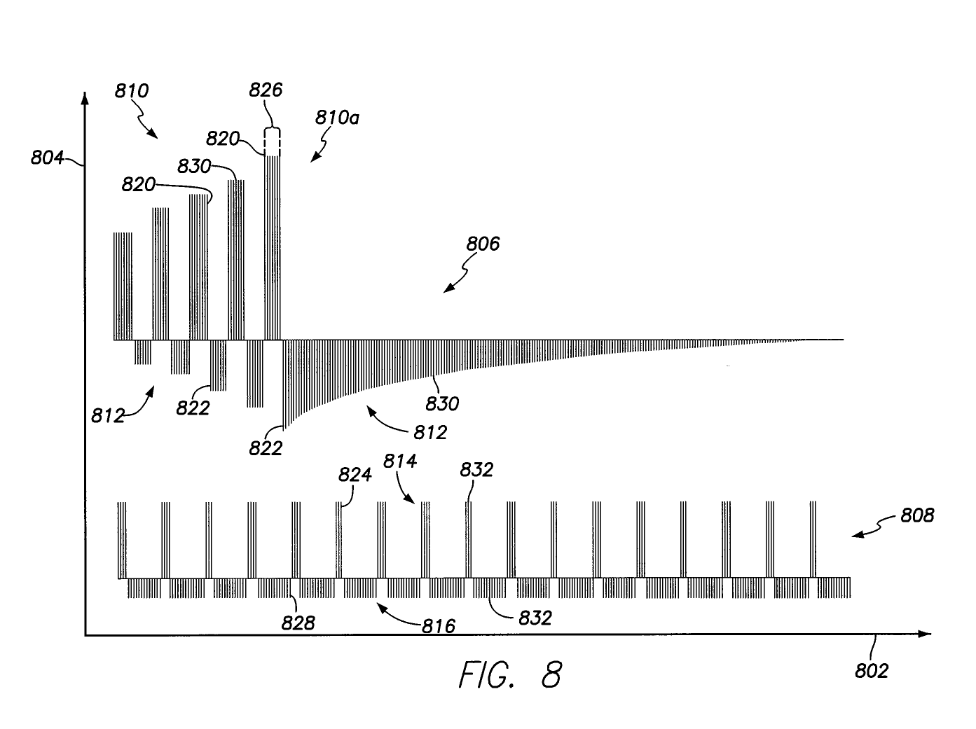

It should be noted, the electrical responses of the membrane of the nerve cells behaves similarly to a low-pass filter, which is described further below in regard to FIGS. 7a-b. When the membrane of the nerve cell is stimulated by the two waveforms 604a and 602, the membrane integrates the two waveforms 604a and 602 together. The integration by the membrane of the two waveforms 604a and 602 allow the nerve cell to be simultaneously stimulated by the buck waveform 602 and tonic waveform 604a even though the pulses by each of the waveforms 604a and 602 are offset. Additionally or alternatively, the stimulation electrodes 111a-d may not be divided into sub-sets and each stimulation electrode 111a-d may emit the two waveforms 604a and/or 602 burst stimulation waveform.

Optionally, the burst and tonic stimulation waveforms may be time multiplexed by subdividing and interleaving pulses (e.g., the recharge pulse 611a/b, the tonic pulse 610, the recharge pulse 608, each of the burst pulses 607) of the tonic and burst stimulation waveforms 602 and 604a into micro pulses 710. For example, FIG. 7a illustrates a pulse 702 with a pulse width 714 and amplitude 716. The horizontal axes 706 represents time and the vertical axes 708 may represent current or voltage. The pulse 702 is subdivided into micro pulses 710 forming a subdivided pulse 704, shown in FIG. 7b, such that over the length of time of the pulse width 714 the micro pulses 710 have a duty cycle of 50%. It should be noted that an amplitude 718 of the micro pulses 710 is shown as twice the amplitude 718 of the pulse 702. The increased amplitude is due to the duty cycle of the micro pulses 710 and the electrical response of the cell membrane of the nerve cell (neuron) (e.g., 712 and 720) receiving the stimulation. The electrical response of the nerve cell 712 to the pulse 702 is shown in FIG. 7a having an exponential increase in charge during the pulse 702 and depolarization 722 after the pulse 702. The electrical response of the nerves to the subdivided pulse 724 is shown in FIG. 7b. During the subdivided pulse 724, the membrane of the nerve cell integrates the subdivided pulse 724 similar to a low pass filter and depolarization 722 after the subdivided pulse 724 at a similar rate as the depolarization 722. Due to the integration of the cell membrane to the subdivided pulse 724 other possible combinations of micro pulse 710 duty cycles and amplitude 718 may be used in alternative embodiments (e.g., 80% duty cycle having an amplitude 1.25 times the amplitude 716, 66% duty cycle having an amplitude 1.5 times the amplitude 716, 33% duty cycle having an amplitude 3 time the amplitude 716, 20% duty cycle having an amplitude 5 times the amplitude 716).

FIG. 8 illustrates a graphical representation of a chopped burst and tonic stimulation waveform 806 and 808 emitted from the stimulation electrodes (e.g., 111a-d, 511a-c). The chopped burst and tonic stimulation waveforms 806 and 808 are time multiplexed, such that, micro pulses 824 and 828 of the chopped tonic stimulation waveform 806 do not occur during micro pulses 820 and 822 of the chopped burst stimulation waveform 808. The chopped burst stimulation waveform 806 includes a series of burst pulses 810 that increase amplitude incrementally. The chopped burst stimulation waveform 806 also includes a series of regeneration pulses 812. The chopped tonic stimulation waveform 808 includes a series of tonic pulses 814 and regeneration pulses 816. Each pulse from the chopped burst and tonic stimulation waveforms 806 and 808 are subdivided into a series of alternating micro pulses 820, 822, 824, and 828. Each alternating micro pulse 820, 822, 824, and 828 may be preceded and/or followed by inactive pulse gaps 830 and 832. During the inactive pulse gaps 830 and 832, the stimulation electrodes may emit current or voltage corresponding to the alternative chopped stimulation waveform 806 and 808. During the gap 830, for example, the stimulation electrodes may emit a micro pulse (e.g., 820, 822) of the burst stimulation waveform 806 between two micro pulses (e.g., 824, 828) of the tonic stimulation waveform 808.

Optionally, the chopped burst and tonic stimulation waveforms 806 and 808 may be emitted from a first sub-set of stimulation electrodes (e.g., 410, 512) and a second sub-set of stimulation electrodes (e.g., 412, 514), respectively. For example, during the inactive pulse gap 830, the first sub-set of stimulation electrodes may not emit current or voltage. Similarly, during the inactive pulse gap 832, the second sub-set of stimulation electrodes may not emit current or voltage.

Optionally, the inactive pulse gap 830 may have the same pulse width as the micro pulses 824 and 828, and/or the inactive pulse gap 832 may have the same pulse width as the micro pulses 820 and 822. For example, the burst pulse 810a has a pulse width 826 of 2 ms. It should be noted that in other embodiments the pulse width 826 may be larger or smaller than 2 ms. The burst pulse 810a is subdivided into a series of alternating micro pulses 820a, such that, each alternating micro pulse 820a is preceded and/or followed by an inactive pulse gap 830. The micro pulses 820a may have a pulse width of 50 microseconds (μs). It should be noted that in other embodiments the micro pulses may be more or less (e.g., 5 μs). The micro pulses 820a subdivide the burst pulse 810a such that the micro pulses 820a occur (e.g., 20 micro pulses 820a) or is active for half of the pulse width 826. Thereby, the micro pulses 820a have a duty cycle of 50%.

It should be noted that in other embodiments the duty cycle of the micro pulses 820, 822, 824, and 828 may be greater than or less than 50%. Optionally, the micro pulses 820, 822, 824, and 828 of the chopped burst and tonic stimulation waveforms 806 and 808, respectively, may have a select duty cycle between 20-80%. Additionally or alternatively, the duty cycles of the micro pulses 820, 822, 824, and/or 820 may not be the same.

Each micro pulse 820 and 822 of the chopped burst stimulation waveform 806 occurs during the inactive pulse gap 832 of the chopped tonic stimulation waveform 808. Additionally, each micro pulse 824 and 828 of the chopped tonic stimulation waveform 808 occurs during the inactive pulse gap 830 of the chopped burst stimulation waveform 806. Similar to the temporal offset described above, the micro pulses 820 and 822 of the chopped burst stimulation waveform 806 do not occur during the micro pulses 824 and 820 of the tonic stimulation waveform 808.

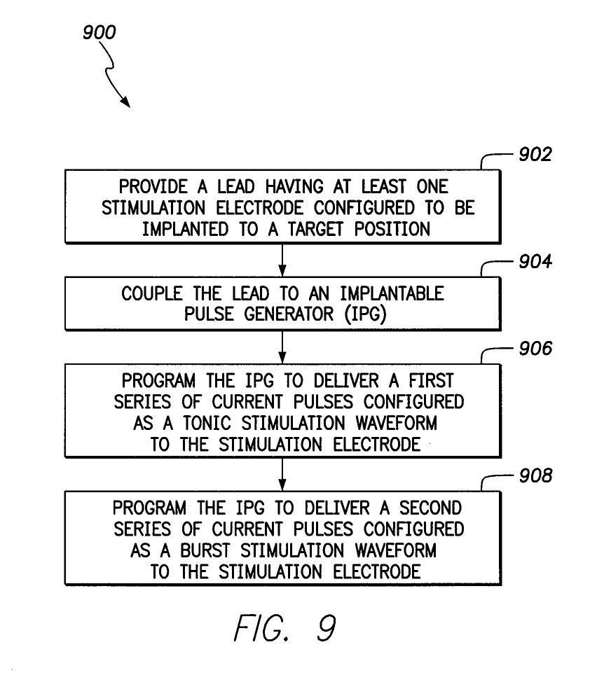

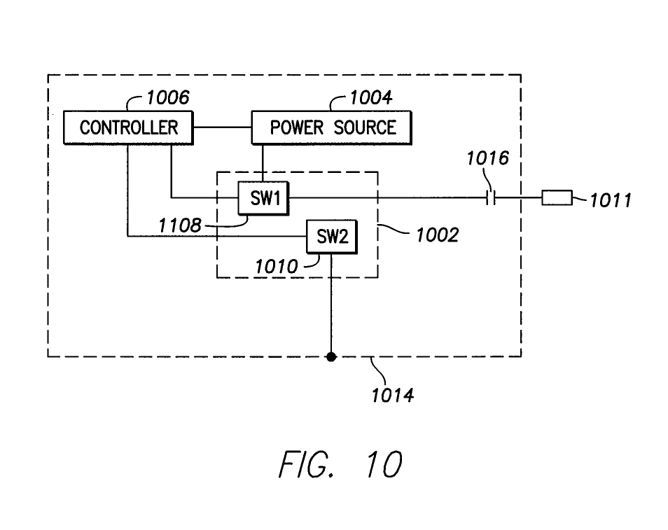

FIG. 9 is a flowchart illustrating a method 900 for simultaneous burst and tonic stimulation of nerve tissue of a patient. The method 900, for example, may employ structures or aspects of various embodiments (e.g., systems and/or methods) discussed herein. For example, an implantable pulse generator (IPG) may be similar to the IPG 150 (FIG. 1) or may include other features, such as those described or referenced herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. Furthermore, it is noted that the following is just one possible method of performing simultaneous burst and tonic stimulation. It should be noted, other methods may be used.

The method 900 includes providing (at 902) a lead 110 having at least one stimulation electrode 111a configured to be implanted to a target position and coupling (at 904) the lead 110 to an implantable pulse generator (IPG) 150. For example, the lead 110 includes stimulation electrodes 111a-d, each of the stimulation electrodes 111a-d are coupled to a blocking capacitor 216a-d. The terminals of one or more leads 110 are inserted within the IPG header of the IPG 150 for electrical connection with respective connectors. Pulses are generated by the IPG and are conducted through IPG header to conductors of the lead 110 and applied to nerve tissue of a patient via stimulation electrodes 111a-d through the blocking capacitors 216a-d. The lead 110 also may be positioned proximate to nerve tissue of interest (e.g., stimulation targets 402 and/or 404).

Optionally, the lead 110 may only include a single stimulation electrode 1011. FIG. 10 is a basic schematic diagram of switching circuitry 1002 for an embodiment of an NS system. The switching circuitry 1002 (e.g., the switching circuitry 157) may be electrically coupled to a controller 1006 (e.g., the controller 151), a power source 1004 (e.g., battery 154), and a blocking capacitor 1016. The blocking capacitor 1016 is electrically coupled to the stimulation electrode 1011. The switching circuitry 1002 is shown with two electrical switches, a switch1 1008 and a switch2 1010. The switches 1008 and 1010 are electrically coupled to the blocking capacitor 1016 and a can 1014 (e.g., the can 158). It should be noted that the switching circuitry 1002 may be generally characterized as switch arrays (e.g., plurality FETS, relays), switch matrixes, or the like. Thereby, in alternative embodiments the switching circuitry 1002 may include more or less switches (e.g., more than two, less than two) than illustrated in FIG. 10. Optionally the switching circuitry 1002 may be integrated within the controller 1006.

The switch1 1008 and switch2 1010 are electrically coupled to the power source 1004 (e.g., the battery 154, boost converter). The power source 1004 provides a direct current or voltage contact for the switch1 1008 and switch2 1010. The switch1 1008 and switch2 1010 are also electrically coupled to a common ground (not shown) for the NS system. The common ground provides a return path for electric current for the NS system. The switch1 1008 and switch2 1010 may direct current or form electrical current paths from the power source 1004 and/or the common ground to the blocking capacitor 1016 and can 1014 by electrically coupling to one of the contacts (e.g., the power source 1004, the common ground). For example, the switch1 1008 may electrically couple the power source 1004 to the blocking capacitor 1016 and the switch2 1010 may electrically couple the can 158 to the common ground. Thereby, the electrode 1011 may receive current or voltage from the power source 1004, which is emitted from the electrode 1011 as a stimulation waveform (e.g., burst stimulation waveform 602, tonic stimulation waveform 604).

The switching circuitry 1002 and the power source 1004 are controlled by the controller 1006 to configure pulses that are emitted from the NS system through the stimulation electrode 1011 and the can 1014. The controller 1006 controls or adjust the amount of current or voltage supplied to the switches 1008 and 1010 by instructing the amount of current or voltage supplied by the power source 1004 to the switches 1008 and 1010. Additionally, the controller 1006 may instruct at least one of the switches 1008 and 1010 to electrically couple to one the blocking capacitor 1016 or the can 1014.

For example, the NS system 100 is programmed to emit a 2 milliampere (mA) pulse. The pulse is programmed to be discharged from the stimulation electrode 1011 in an anode state or when the stimulation electrode 1011 is electrically coupled to the power source 1008 via the switch1 1008, and grounded by the can 1014 in a cathode state or when the can 1014 is electrically coupled to the common ground via the switch2 1010. The controller 1006 may instruct the power source 1004 to supply a 2 mA supply current to the switches 1008 and 1010. The controller 1006 may instruct the switch1 1008 to direct current or electrically couple the blocking capacitor 1016 to the power source 1004. The controller 1006 may further instruct the switch2 1010 to electrically couple the can 1014 to the common ground. The controller 1006 may increase the amplitude of the pulse, for example, to have the electrode 1011 emit a pulse corresponding to an alternative stimulation waveform (e.g., from the tonic pulse 610 to the burst pulses 607).

Additionally or alternatively, the controller 1006 may switch the polarity of the pulse to deliver a recharge pulse to maintain charge balance on the blocking capacitor 1016. For example, the controller 1006 may instruct the switch1 1010 to electrically couple the blocking capacitor 1016 to the common ground. The controller 1006 may further instruct the switch2 1010 to electrically couple the can 1014 to the power source 1004.

Returning to FIG. 9, the method 900 includes programming (at 906) the IPG 150 to deliver a first series of current pulses (e.g., tonic pulse 610 and recharge pulse 611a) configured as the tonic stimulation waveform 604a to the stimulation electrode 111a and programming (at 908) the IPG 150 to deliver a second series of current pulses (e.g., burst pulses 607 and recharge pulse 608) configured as the burst stimulation waveform 602 to the stimulation electrode 111a. For example, the IPG 150 may be programmed or receive stimulation programs from the controller device 160. The stimulation program may include current pulse specifications to deliver each stimulation waveform 604a and 608. Further, the current pulse specifications for each stimulation waveform 604a and 608 may include having at least two current pulses of different amplitude polarities (e.g., tonic pulse 610 and recharge pulse 611a, the burst pulses 607 and recharge pulse 608). Additionally, the stimulation program may have the switching circuitry 157 have each stimulation waveform 604a and 608 emitted through two different sub-sets of stimulation electrodes (e.g., 410 and 412). Optionally, each sub-set of stimulation electrodes (e.g., 510 and 512) may have only one unique stimulation electrode (e.g., 511b).

In an embodiment, the method 900 may include programming the IPG 150 to deliver a third series of current pulses configured as another burst stimulation waveform to the stimulation electrodes. For example, FIG. 11 illustrates a graphical representation of two chopped burst stimulation waveforms 1106 and 1108 and a chopped tonic stimulation waveform 1110 emitted from the stimulation electrodes. The chopped burst stimulation waveforms 1106, 1108, and 1110 are time multiplexed, such that, only one micro pulse 1112, 1114, or 1118 occurs at a time. Optionally, the IPG 150 may deliver the three stimulation electrodes to a first, second, and third sub-set of stimulation electrodes, respectively. Additionally or alternatively, each sub-set of stimulation electrodes may have at least one unique stimulation electrode relative to each other.

The chopped burst stimulation waveforms 1106 and 1108 include a series of burst pulses 1120 and 1122. The chopped burst stimulation waveforms 1106 and 1108 also include a series of regeneration pulses 1124 and 1126 with a different amplitude polarity than the burst pulses 1120 and 1122. The chopped tonic stimulation waveform 1110 includes a series of tonic pulses 1128 and regeneration pulses 1130 with a different amplitude polarity. Each pulse from the chopped burst and tonic stimulation waveforms 1106, 1108, and 1110 are subdivided into a series of alternating micro pulses 1112, 1114 and 1118 such that each of the micro pulses 1112, 1114, and 1118 has a duty cycle of 33%. Each alternating micro pulse 1112, 1114 and 1118 may be preceded and/or followed by an inactive pulse 1132, 1134, and 1136, respectively.

The controllers 151, 206, 1006 and the controller device 160 may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the controllers 151, 206, 1006 and the controller device 160 may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The controllers 151, 206, 1006 and the controller device 160 may execute a set of instructions that are stored in one or more storage elements, in order to process data. The storage elements may also store data or other information as desired or needed. The storage element may be in the form of an information source or a physical memory element within the controllers 151, 206, 1006 and the controller device 160. The set of instructions may include various commands that instruct the controllers 151, 206, 1006 and the controller device 160 to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112(f), unless and until such claim limitations expressly use the phrase “means for” followed by a statement of function void of further structure.

What is claimed is:

1. A method for simultaneous burst and tonic stimulation of nerve tissue of a patient, the method comprising:
providing a lead having at least one stimulation electrode on the lead configured to be implanted at a target position proximate to nerve tissue of interest;
coupling the lead to an implantable pulse generator (IPG) such that current pulses are generated by the IPG and delivered through blocking capacitors to the stimulation electrodes;
programming the IPG to deliver a first series of current pulses configured as a tonic stimulation waveform to the stimulation electrode and to deliver a second series of current pulses configured as a burst stimulation waveform to the stimulation electrode, wherein the tonic stimulation waveform and the burst stimulation waveform each include at least two current pulses having different amplitude polarities.

2. The method of claim 1, wherein the current pulses of the tonic stimulation waveform and the current pulses of the burst stimulation waveform are temporally offset with respect to each other such that the current pulse of the tonic stimulation waveform does not occur during the current pulse of the burst stimulation waveform.

3. The method of claim 1, wherein the tonic stimulation waveform occurs within the burst stimulation waveform such that current pulses of the burst stimulation waveform occurs before and after the tonic stimulation waveform.

4. The method of claim 1, wherein the tonic stimulation waveform and the burst stimulation waveform are charge balanced such that a voltage potential across the blocking capacitors of the stimulation electrodes are approximately the same before and after the waveforms are discharged from the blocking capacitors.

5. The method of claim 1, wherein the tonic stimulation waveform and the burst stimulation waveform are chopped waveforms such that each current pulse is sub-divided into micro pulses with a select duty cycle between 20-80%.

6. The method of claim 5, wherein each micro pulse of the tonic stimulation waveform does not occur during micro pulses of the burst stimulation waveform.

7. The method of claim 5, wherein at least one of the micro pulses of the burst stimulation waveform is between two micro pulses of the tonic stimulation waveform.

8. The method of claim 1, wherein there is a plurality of stimulation electrode; and
wherein the programming operation includes the IPG delivering the tonic stimulation waveform to a first sub-set of the stimulation electrodes and the burst stimulation waveform to a second sub-set of the stimulation electrodes, the first and second sub-sets have at least one unique stimulation electrode relative to each other.

9. The method of claim 1, wherein the tonic stimulation waveform is emitted towards a first region of nervous tissue and the burst stimulation waveform is emitted towards a second region of nervous tissue.

10. The method of claim 1, further comprising programming the IPG to deliver a third series of current pulses configured as another burst stimulation waveform to the stimulation electrodes, wherein the other burst stimulation waveform includes at least two current pulses having different amplitude polarities.

11. A system for simultaneous burst and tonic stimulation, the system comprising:
a lead having at least one stimulation electrode, the lead configured to be implanted at a target position proximate to or within nerve tissue of interest; and an implantable pulse generator (IPG) coupled to the lead, the IPG configured to deliver a first and second series of current pulses through the blocking capacitors to the stimulation electrodes, wherein the first series of current pulses are configured as a tonic stimulation waveform, the second series of current pulses are configured as a burst stimulation waveform, and the tonic stimulation waveform and the burst stimulation waveform each include at least two current pulses having different amplitude polarities.

12. The system of claim 11, wherein the lead includes a plurality of stimulation electrode, and the IPG is configured to deliver the tonic stimulation waveform to a first sub-set of the stimulation electrodes and the burst stimulation waveform to a second sub-set of the stimulation electrodes, the first and second sub-sets have at least one unique stimulation electrode relative to each other.

13. The system of claim 11, wherein the current pulses of the tonic stimulation waveform and the current pulses of the burst stimulation waveform are temporally offset with respect to each other such that the current pulse of the tonic stimulation waveform does not occur during the current pulse of the burst stimulation waveform.

14. The system of claim 11, wherein the tonic stimulation waveform occurs within the burst stimulation waveform such that current pulses of the burst stimulation waveform occurs before and after the tonic stimulation waveform.

15. The system of claim 11, wherein the tonic stimulation waveform and the burst stimulation waveform are charge balanced such that a voltage potential across the blocking capacitors of the stimulation electrodes are approximately the same before and after the waveforms are discharged from the blocking capacitors.

16. The system of claim 11, wherein the tonic stimulation waveform and the burst stimulation waveform are chopped waveforms such that each current pulse is sub-divided into micro pulses with a select duty cycle between 20-80%.

17. The system of claim 16, wherein each micro pulse of the tonic stimulation waveform does not occur during micro pulses of the burst stimulation waveform.

18. The system of claim 16, wherein at least one of the micro pulses of the burst stimulation waveform is between two micro pulses of the tonic stimulation waveform.

19. The system of claim 11, wherein none of the stimulation electrodes of the first sub-set are included within the second sub-set.

20. The system of claim 11, wherein the tonic stimulation waveform is emitted towards a first region of nervous tissue and the burst stimulation waveform is emitted towards a second region of nervous tissue.
